SEQUENCE LISTING (5 sequences)

SEQ ID NO: 1 - protein, 458 aa, Homo sapiens
  MISC_FEATURE (152)..(152): X At position 152 may be Glycine or Glutamate
LERGRDYEKNKVCKEFSHLGKEDFTSLSLVLYSRKFPSGTFEQVSQLVKEVVSLTEACCAEGADPDCYDTRTSALSAKSCESNSPFPVHPGTAECCTKEGLERKLCMAALKHQPQEFPTYVEPTNDEICEAFRKDPKEYANQFMWEYSTNYXQAPLSLLVSYTKSYLSMVGSCCTSASPTVCFLKERLQLKHLSLLTTLSNRVCSQYAAYGEKKSRLSNLIKLAQKVPTADLEDVLPLAEDITNILSKCCESASEDCMAKELPEHTVKLCDNLSTKNSKFEDCCQEKTAMDVFVCTYFMPAAQLPELPDVELPTNKDVCDPGNTKVMDKYTFELSRRTHLPEVFLSKVLEPTLKSLGECCDVEDSTTCFNAKGPLLKKELSSFIDKGQELCADYSENTFTEYKKKLAERLKAKLPDATPTELAKLVNKXSDFASNCCSINSPPLYCDSEIDAELKNIL

SEQ ID NO: 2 - protein, 458 aa, Homo sapiens
  MISC_FEATURE (152)..(152): X at position 152 may be Glycine or Glutamate
LERGRDYEKNKVCKEFSHLGKEDFTSLSLVLYSRKFPSGTFEQVSQLVKEVVSLTEACCAEGADPDCYDTRTSALSAKSCESNSPFPVHPGTAECCTKEGLERKLCMAALKHQPQEFPTYVEPTNDEICEAFRKDPKEYANQFMWEYSTNYXQAPLSLLVSYTKSYLSMVGSCCTSASPTVCFLKERLQLKHLSLLTTLSNRVCSQYAAYGEKKSRLSNLIKLAQKVPTADLEDVLPLAEDITNILSKCCESASEDCMAKELPEHTVKLCDNLSTKNSKFEDCCQEKTAMDVFVCTYFMPAAQLPELPDVELPTNKDVCDPGNTKVMDKYTFELSRRTHLPEVFLSKVLEPTLKSLGECCDVEDSTTCFNAKGPLLKKELSSFIDKGQELCADYSENTFTEYKKKLAERLKAKLPEATPTELAKLVNKXSDFASNCCSINSPPLYCDSEIDAELKNIL

SEQ ID NO: 3 - protein, 458 aa, Homo sapiens
  MISC_FEATURE (152)..(152): X at position 152 may be Glycine or Glutamate
LERGRDYEKNKVCKEFSHLGKEDFTSLSLVLYSRKFPSGTFEQVSQLVKEVVSLTEACCAEGADPDCYDTRTSALSAKSCESNSPFPVHPGTAECCTKEGLERKLCMAALKHQPQEFPTYVEPTNDEICEAFRKDPKEYANQFMWEYSTNYXQAPLSLLVSYTKSYLSMVGSCCTSASPTVCFLKERLQLKHLSLLTTLSNRVCSQYAAYGEKKSRLSNLIKLAQKVPTADLEDVLPLAEDITNILSKCCESASEDCMAKELPEHTVKLCDNLSTKNSKFEDCCQEKTAMDVFVCTYFMPAAQLPELPDVELPTNKDVCDPGNTKVMDKYTFELSRRTHLPEVFLSKVLEPTLKSLGECCDVEDSTTCFNAKGPLLKKELSSFIDKGQELCADYSENTFTEYKKKLAERLKAKLPDATPKELAKLVNKXSDFASNCCSINSPPLYCDSEIDAELKNIL

SEQ ID NO: 4 - protein, 83 aa, Artificial Sequence
  Synthetic Peptide
LLKKELSSFIDKGQELCADYSENTFTEYKKKLAERLKAKLPDATPTELAKLVNKXSDFSNCCSINSPPLYCDSEIDAELKNIL

SEQ ID NO: 5 - protein, 84 aa, Artificial Sequence
  Synthetic peptide
LLKKELSSFIDKGQELCADYSENTFTEYKKKLAERLKAKLPDATPKELAKLVNKXSDFASNCCSINSPPLYCDSEIDAELKNIL

(19) United States
(12) Patent Application Publication
(10) Pub. No.: US 2014/0030215 A1
(43) Pub. Date: Jan. 30, 2014

(54) Macrophage Activating Factor For Pharmaceutical Compositions

(71) Applicant: Nobuto Yamamoto, Philadelphia, PA (US)

(72) Inventor: Nobuto Yamamoto, Philadelphia, PA (US)

(21) Appl. No.: 14/047,771

(22) Filed: Oct. 7, 2013

Related U.S. Application Data
(63) Continuation of application No. PCT/IL2012/000159, filed on Apr. 5, 2012.
(60) Provisional application No. 61/472,642, filed on Apr. 7, 2011.

(51) Int. Cl. A61K 38/19

(52) U.S. Cl. CPC A61K 38/19
USPC 424/85.1; 530/351; 435/68.1

(57) ABSTRACT

Pharmaceutical compositions including macrophage activating factor (MAF) and method of producing same, particularly to MAF compositions essentially devoid of glycosidase enzymes. The compositions of the present invention and pharmaceutical compositions including same are particularly suitable for intravenous administration.

### FIELD OF THE INVENTION

The present invention relates to pharmaceutical compositions comprising Gc protein-derived macrophage activating factor (GcMAF) and method of producing same, particularly to GcMAF compositions essentially devoid of glycosidase enzymes.

### BACKGROUND OF THE INVENTION

Inflammation results in macrophage activation, leading to a progress in the immune response process. Inflammation-derived macrophage activation requires participation of B and T lymphocytes and serum vitamin D binding protein (DBP).

The human vitamin D-binding protein, also known as “group-specific component” or “Gc protein”, is an evolutionary conserved glycoprotein showing genetic polymorphism. It is a plasma protein having a relative molecular weight of about 52,000, normally constituting about 0.5% of the plasma proteins in human. Polymorphism of the Gc protein is demonstrable by gel electrophoresis analysis, which reveals two major phenotypes: Gc1 and Gc2 (Hirschfeld et al., 1960. Nature 185:931). The entire nucleotide coding sequences of the Gc1 and Gc2 genes, and the predicted amino acid sequences have been reported (Cooke, et al., 1985. J. Clin. Invest. 76:2420; Yang et al., 1985. Proc. Natl. Acad. Sci. USA 82:7994). Gc1 is further divided into Gc1f and Gc1s subtypes, which migrate electrophoretically as two bands (“fast” and “slow”), due to a variation in one amino acid residue.

Gc1 protein is the major subtype of human Gc protein. It carries a branched trisaccharide composed of N-acetylgalactosamine (GalNAc) attached to the core protein with a termini of galactose and sialic acid (in Gc1f) or galactose and mannose (in Gc1s). Gc2 has a simple glycosylation pattern with a core GalNAc linked to a terminal galactose moiety. Gc1f oligosaccharide is hydrolyzed by membranous β-galactosidase of inflammation-primed B cells to yield a macrophage pro-activating factor, which is in turn hydrolyzed by sialidase (also known as neuraminidase) of T-cells to yield a macrophage activating factor (MAF) (Yamamoto et al., 1991. Proc. Nail. Acad. Sci. USA 88:8539-8543; Yamamoto and Kumashiro 1993. Immunol, 151:2794-2902; Naraparaju and Yamamoto 1994. Immunol. Lets 43:143-148). Mouse DBP carries a disaccharide composed of N-acetylgalactosamine with a galactose terminal. Hydrolysis of this disaccharide by β-galactosidase of B cells alone generates a potent MAF. Thus, mouse DBP and human Gc protein are precursors for MAFs.

U.S. Pat. No. 5,177,002 to the inventor of the present invention discloses a process for in vitro production of potent macrophage activating factor by treating the glycosylated vitamin D-binding protein with glycosidases. The phenotypes of Gc protein Gc1 and Gc2, and the Gc1 subtypes Gc1f and Gc1s, are expressed inter alia as differences in the oligosaccharides attached to the polypeptide portion of the Gc molecule. The macrophage activating factor is efficiently produced from Gc1f or Gc1s protein by incubation with a combination of β-galactosidase and sialidase (also known as neuraminidase), or a combination of β-galactosidase and α-mannosidase. If the Gc1s comprises at least in part the Gc1s variant, Gc1s*, which contains sialic acid (N-acetyl-D-neuramic acid, or “NeuNAc”) in lieu of α-mannose, the mixture of enzymes utilized to treat the Gc1s/Gc1s* mixture also includes sialidase. Treatment of the Gc2 protein with β-galactosidase alone efficiently yields the macrophage activating factor. Thus, the U.S. Pat. No. 5,177,002 discloses efficient conversion of Gc protein to the macrophage activating factor in vitro, in the absence of intact B- and T-cells, resulting in a highly potent factor designated GcMAF.

The uncontrolled growth of metastases resistant to conventional therapeutic modalities is a major cause of death from cancer. Metastases arise from the nonrandom spread of specialized malignant cells that preexist within a primary neoplasm. These metastases can be clonal in their origin, and different metastases can originate from different progenitor cells. In addition, metastatic cells can exhibit an increased rate of spontaneous mutations compared with benign non-metastatic cells, which explain the clinical observation that multiple metastases can exhibit different sensitivity to the same therapeutic modalities. A successful therapy of disseminated metastases thus should circumvent the problems of neoplastic heterogeneity and development of resistance.

Appropriately activated macrophages can fulfill these demanding criteria. Macrophages can be activated to become tumoricidal by interaction with phospholipid vesicles (liposomes) containing immunomodulators. Tumoricidal macrophages can recognize and destroy neoplastic cells in vitro and in vivo, leaving non-neoplastic cells uninjured. Although the exact mechanism(s) by which macrophages discriminate between tumorigenic and normal cells is unknown, it is independent of tumor cell characteristics such as immunogenicity, metastatic potential, and sensitivity to cytotoxic drugs. Moreover, macrophage destruction of tumor cells apparently is not associated with the development of tumor cell resistance. Additionally, activated macrophages are essential for the immune response to form relative to bacterial and viral invasion. As the mechanism of activation is identical in the three responses (tumoricidal, bactericidal, viricidal), the activation of macrophages has applications across the host immune response, against tumor, bacteria and viral challenges.

Studies have shown that GcMAF has a tumoricidal role in the treatment of an Ehrlich ascites tumor in a mouse model (Yamamoto et al., 1997. Cancer Res. 57:2187-2192; Koga et al., 1999. Proc Soc Exp Biol Med. 220:20-26). In a mice model of squamous cell carcinoma, administration of GcMAF as an adjuvant immunotherapy to photodynamic therapy showed a synergistic effect on tumor cure in mice (Korbelik et al. 1997.Br J Cancer 75:202-207). In both tumor models, it was hypothesized that GcMAF elicited its effect by activating macrophages, which then directly attacked the tumor cells. Further evidence suggested that GcMAF is anti-tumorigenic in part through an antiangiogenic mechanism (Kisker et al., 2003. Neoplasia 5(1):32-40). In all models, high potency of GcMAF as an anti-tumorigenic therapy was observed.

U.S. Patent Application Publication No. 2011/0123591 discloses methods of inducing a tumoricidal, bactericidal or viricidal response in a mammal by macrophage activation through the use of an extracorporeal system. The system comprises means for contacting a leukocyte fraction of the mammal's blood with GcMAF or with one or more enzymes that create endogenous GcMAF from Gc protein precursor. Alternatively, the system comprises alpha-N-acetylgalactosaminidase (Nagalase)-binding ligand immobilized on an inert medium that is contacted with the mammal's plasma, thus reducing the level of Nagalase.

The precursor of MAF, the glycosylated Gc protein, can be purified from blood source. Alternatively, Gc protein or its small domain responsible for macrophage activation can be produced employing recombinant methods, as disclosed, for example, in U.S. Pat. No. 6,410,269 to the inventor of the present invention. Independent of the Gc protein source, it should be partially deglycosylated in order to obtain the macrophage activating factor as described hereinabove. The U.S. Pat. No. 5,177,002 discloses the use of immobilized enzymes and passing the reaction mixture through an appropriate cut-off filter to avoid contamination of the final preparations with glycosidases. No particular guidelines are provided for selecting the immobilization means, with activated agarose beads being the preferred embodiments.

As of today, the use of GcMAF has been limited to ex vivo treatments or to intramuscular administrations in order to avoid adverse side effects, apparently due to the presence of contaminating proteins in the compositions produced as per the procedures previously disclosed.

As the potential of GcMAF as a potent therapy for immuno-deficient diseases (such as AIDS), various types of cancer, viral infections and osteopetrosis has been established, it would be highly advantageous to have GcMAF composition devoid of protein contamination, suitable for intravenous administration.

### SUMMARY OF THE INVENTION

The present invention relates to therapeutic Gc-protein derived macrophage activating factor (GcMAF) compositions and methods of producing same.

The teachings of the present invention overcomes the shortages of hitherto disclosed GcMAF compositions, which were found to contain residues of the glycosidase enzymes used for transforming Gc protein to macrophage activating factor, and thus may cause adverse effects when administered pharmaceutically, particularly when administered intravenously.

The present invention is based in part on the unexpected finding that GcMAF preparations obtained from contacting Gc protein source with β-galactosidase immobilized on polygalactose-based resins (e.g. sepharose beads), contained deleterious amounts of the β-galactosidase enzyme. Without wishing to be bound by any specific theory or mechanism of action, the presence of the glycosidase residues found in the GcMAF compositions may be due to the phenomenon of solid support digestion by the glycosidase enzymes attached thereto.

Thus, according to one aspect, the present invention provides a composition comprising Gc protein-derived macrophage activating factor (GcMAF), wherein the composition is essentially devoid of glycosidase enzymes.

As used herein the term “essentially devoid of glycosidase enzymes” refers to a composition containing less than 3% glycosidase enzymes of the composition total protein content, typically less than 2% or 1%, more typically less than 0.5% or 0.2% of the total protein content of said composition.

According to certain embodiments, the GcMAF comprises vitamin D-binding protein (Gc Protein) or an active fragment thereof having an N-acetylgalactosamine group linked to an amino acid residue.

The terms “vitamin D-binding protein” and “Gc protein” are used herein interchangeably and refer to all the polymorphic forms of the glycoprotein and genetic variations thereof, including Gc2, Gc1, and the subtypes Gc1f, Gc1s and Gc1s*. As used herein the term “an active fragment thereof” refers to any part of Gc protein having a terminal N-acetylgalactosamine group linked to an amino acid residue, capable of macrophage activation.

According to certain embodiments, the Gc protein fragment comprises the amino acid sequence corresponding to amino acids 400-435 of all mature Gc protein polymorphic forms. According to other embodiments, the Gc fragment is Gc protein domain III corresponding to amino acids 375-458 of the mature protein.

According to certain embodiments, the Gc protein comprises the amino acid sequence selected from the group consisting of SEQ ID NOs:1-3 (Gc1f, Gc1s and Gc2, respectively). According to these embodiments, the N-acetylgalactosamine group is linked to the amino acid threonine at position 418 or amino acid threonine at position 420 of the mature Gc protein.

According to other embodiments, the Gc fragment Domain III, corresponding to amino acids 375-458 of the mature Gc protein consist of the amino acid sequence set forth in either SEQ ID NO:4 or SEQ ID NO:5. According to this embodiment, the N-acetylgalactosamine is linked to the amino acid threonine at position 44 or amino acid threonine at position 46.

According to certain embodiments, the isolated vitamin D-binding protein or any part thereof is purified from human blood serum. According to other embodiments, the isolated vitamin D-binding protein or its small domain responsible for macrophage activation can be produced from cloned polynucleotides employing recombination systems.

Removal of the glycosidase enzymes transforming the Gc protein to macrophage activating factor from the composition medium may be performed by any method as is known to a person skilled in the art, including, but not limited to, affinity chromatography, ion-exchange chromatography and gel filtration. According to certain typical embodiments, the enzymes are immobilized on a solid phase, particularly a solid phase devoid of the enzyme substrate.

Immobilizing the enzymes on a solid support has several advantages, including simple separation of the enzymes from the desired protein(s). The present invention now discloses that the use of solid phase devoid of the enzyme substrate prevents release of soluble enzyme into the reaction medium, facilitating the removal of the non-desired enzymes from the final composition.

Thus, according to additional aspect, the present invention provides a process for producing a GcMAF composition essentially devoid of glycosidase enzymes, the process comprising (a) contacting Gc protein or an active fragment thereof in vitro with the glycosidase enzyme β-galactosidase or with β-galactosidase in combination with at least one additional glycosidase enzyme, wherein each of the glycosidase enzymes is immobilized on a solid phase devoid of said enzyme substrate, to obtain Gc-macrophage activating factor (GcMAF); and (b) removing the immobilized enzyme from the GcMAF composition, thereby obtaining a GcMAF composition essentially devoid of glycosidase enzymes.

According to certain embodiments, the additional glycosidase enzyme is selected from the group consisting of mannosidase and sialidase.

According to some embodiments, the Gc protein is Gc1f and is contacted with β-galactosidae and sialidase (Neuraminidase).

According to certain embodiment, the process further comprises subjecting the GcMAF containing composition to ion exchange chromatography. According to typical embodiments, the chromatography is anion exchange chromatography, using, for examples Q-sepharose column. According to alternative embodiments, the process further comprises subjecting the GcMAF composition to hydrophobic interaction chromatography, for example phenyl sepharose column. According to other embodiment, the process further comprises combination of ion exchange chromatography and hydrophobic interaction chromatography.

According to certain embodiments, the β-galactosidase is immobilized on a solid phase comprising acrylic beads. According to particular embodiments, the solid phase is hydrophilic acrylic beads.

According to certain embodiments Gc protein of phenotype Gc1, subtype Gc1f, is contacted with immobilized β-galactosidase and immobilized sialidase to provide the macrophage activating factor. According to other embodiments, Gc Protein of phenotype Gc1, subtype Gc1s, is contacted with immobilized β-galactosidase and at least one immobilized additional glycosidase as required. According to certain typical embodiments, Gc Protein of phenotype Gc1, subtype Gc1s, is contacted with a combination of β-galactosidase and mannosidase and sialidase, each immobilized on a solid phase devoid of the enzyme substrate, to ensure the conversion of the Gc1s variant (h Gc1s*) which contains sialic acid in lieu of α-mannose. According to additional embodiments, Gc protein of phenotype Gc2 is contacted with immobilized β-galactosidase alone to form the macrophage activating factor.

According to another aspect, the present invention provides macrophage activating factor prepared according to the process described hereinabove. According to certain embodiments, the macrophage activating factor is essentially devoid of glycosidase enzymes.

According to additional aspect, the present invention provides a pharmaceutical composition comprising a therapeutically effective amount the macrophage activating factor of the present invention, further comprising pharmaceutically acceptable diluent or carrier. According to preferred embodiments, the pharmaceutical composition is formulated for intravenous administration.

According to yet further aspect the present invention provides a method for inducing macrophage activation in an individual in need thereof comprising administering to the individual a pharmaceutical composition comprising the macrophage activating factor of the invention.

According to yet additional aspect, the present invention provides a method for treating cancer or HIV-infected patients, comprising administrating to a subject in need thereof a therapeutically effective amount of pharmaceutical composition comprising the GcMAF of the invention.

According to certain embodiments, the GcMAF is administered at a dose of 100-500 ng/injection

According to certain embodiments, the cancer is associated with elevated levels of alpha-N-acetylgalactosaminidase (Nagalase). According to other embodiments, the cancer is selected from the group consisting of breast cancer, prostate cancer, colorectal cancer, liver cancer, lung cancer, head/neck cancer, brain cancer, kidney cancer, bladder cancer, stomach cancer, uterus cancer, ovarian cancer, skin cancer, fibrosarcoma, mesothelioma, leukemia and melanoma.

Other objects, features and advantages of the present invention will become clear from the following description and drawings.

### DETAILED DESCRIPTION OF THE INVENTION

The present invention provides therapeutic compositions comprising Gc protein-derived macrophage activating factor. The compositions of the invention are advantageous over hitherto known compositions, being devoid of contaminating enzymes and suitable for intravenous administration.

Gc protein, also designated vitamin D binding protein, is a serum factor comprising a polypeptide with specific oligosaccharides attached thereto. Step-wise removal of certain oligosaccharides with specific glycosidase enzymes results in transforming the Gc protein to highly potent macrophage activating factor (MAF) as disclosed in U.S. Pat. No. 5,177,002 to the inventor of the present invention, incorporated herein in its entirety by reference.

Human Gc protein can be purified from blood serum by any method as is known to a person skilled in the art. According to certain embodiments, Gc protein of high purity for use in the process of the invention is isolated from blood serum using 25-hydroxyvitamin D₃-Sepharose affinity chromatography according to the procedure of Link et al. (1986. Anal. Biochem. 157:262). The Gc protein may also be purified by actin-agarose affinity chromatography according to the procedure of Haddad et al. (1984. Biochem. J. 218:805), which takes advantage of the binding specificity of Gc protein for actin.

Alternatively, the Gc protein can be obtained from cloned cDNA encoding the human Gc protein or Gc protein small domain (domain Ill). Cloning and expression of Gc protein and Gc domain III was described in U.S. Pat. No. 6,410,269 to the inventor of the present invention, incorporated herein in its entirety by reference. The method described therein employs a human liver cDNA library in bacteriophage λgt11 (Clontech, Palo Alto, Calif.) for isolating a full length cDNA encoding the human Gc protein, and the use of the baculoviral expression system in insect cells for the protein expression. However, it is to be explicitly understood that any method/system as is known in the art can be used for expressing a cDNA encoding the Gc protein or active part thereof, including bacterial, insect and mammalian cell systems. According to certain embodiments, expression is performed in a eukaryotic cell such that the Gc protein or its active domain is correctly glycosylated. Any such cell system known in the art may be used, for example Chinese hamster ovary (CHO) cells, BHK cells, human embryonic kidney HEK293 cells and Saccharomyces cerevisiae. Accordingly, any eukaryotic expression vector can be used, including, but not limited to, pCI-NEO, pWE3, pcDNA3.1 and pCM182. Insertion of the vector into the selected cell system can be performed, for example, by electroporation, lipids such as TransFectin or chemical methods as is known to a person skilled in art, with or without amplification. The transfection may result is transient or stable expression, both forms being adequate to obtain the desired Gc protein or part thereof. The expressed protein, being the precursor of active MAF according to the teaching of the present invention, can then be extracted from the cells or collected from the growth media by any method known in the art.

The term “Gc protein” or vitamin D-binding protein” as used herein refer to all genotypes, including Gc2, Gc1, and the subtypes Gc1f, Gc1s and Gc1s* and active variants and fragments thereof. “Active” Gc protein or fragment thereof as used herein refers to Gc protein capable of activating macrophages, particularly to Gc protein or a fragment thereof having an N-acetylgalactosamine group linked to an amino acid residue.

According to certain embodiments, the Gc protein comprises the amino acid sequence selected from the group consisting of SEQ ID NOs:1-3 (Gc1f, Gc1s and Gc2, respectively). According to these embodiments, the N-acetylgalactosamine group is linked to the amino acid threonine at position 418 or amino acid threonine at position 420 of the mature Gc protein.

According to certain embodiments, the Gc protein fragment comprises the amino acid sequence corresponding to amino acids 400-435 of all mature Gc protein polymorphic forms. According to other embodiments, the Gc fragment is Gc protein domain III corresponding to amino acids 375-458 of the mature protein.

According to other embodiments, the Gc fragment Domain III, corresponding to amino acids 375-458 of the mature Gc protein consist of the amino acid sequence set forth in either SEQ ID NO:4 or SEQ ID NO:5. According to this embodiment, the N-acetylgalactosamine is linked to the amino acid threonine at position 44 or amino acid threonine at position 46.

Macrophages have a potential to eliminate cancerous cells and HIV-infected cells when activated. Deglycosylation of GcMAF by the enzyme alpha-N-acetylgalactosaminidase (Nagalase) prevents it from activating macrophages and therefore suppresses the cell immune response. In HIV infected patients it has been suggested that defective antigen presentation is a factor in immune deficiency. The presence of elevated Nagalase in the plasma of HIV patients suggests that macrophage activation may be inhibited in these patients. In addition, Nagalase has been shown to be an intrinsic component of an envelope protein promoting fusion for the initiation of infection. The plasma concentration of Nagalase in patients with systemic lupus erythematosus was also found to be elevated. In lupus, autoantibodies form pathogenic immune complexes and are deposited in tissues. The clearance of these complexes by macrophages is inhibited if macrophage activation is disrupted. Cancer cells have been shown to produce Nagalase and elevated concentrations in serum have been recorded in a number of cancer patients suffering from melanoma, prostate, colorectal, and metastatic breast cancer. Administering exogenous GcMAF to such patients may thus overcome the shortage in active macrophages due to the elevated concentrations of Nagalase. Indeed, GcMAF has been shown to act directly and activate macrophages or osteoclasts of cancer, HIV-infected and osteoperotic patients, and preliminary clinical trails showed a curative effect of GcMAF on several types of human cancer (e.g. Pacini S. et al., 2012. Anticancer Res.;32(1):45-52; Gregory KJ et al. 2010. PLoS One. 2010 5(10):e13428).

To be suitable for pharmaceutical use, particularly when formulated for intravenous administration, the GcMAF composition should stand meticulous requirements of being non-toxic and highly tolerable by human.

Accordingly, the glycosidase enzymes required for transforming Gc protein or Domain III thereof to active MAF as well as potential other contaminant should be removed from the MAF composition.

According to one aspect, the present invention provides a composition comprising Gc protein-derived macrophage activating factor (GcMAF), wherein the composition is essentially devoid of glycosidase enzymes.

According to additional aspect, the present invention provides a process for producing a GcMAF composition essentially devoid of glycosidase enzymes, the process comprising (a) contacting Gc protein or an active fragment thereof in vitro with the glycosidase enzyme β-galactosidase or with β-galactosidase in combination with at least one additional glycosidase enzyme, wherein each of the glycosidase enzymes is immobilized on a solid phase devoid of said enzyme substrate, to obtain Gc-macrophage activating factor (GcMAF); and (b) removing the immobilized enzyme from the GcMAF composition, thereby obtaining a GcMAF composition essentially devoid of glycosidase enzymes.

Removal of the glycosidase enzymes may be performed by any method as is known in the art. According to certain typical embodiments of the invention, the Gc protein or part thereof is contacted with the enzyme(s) wherein the enzymes are immobilized on a solid phase, wherein the solid phase is devoid of the enzyme substrate. Of particular importance is the immobilization of β-galactosidase on a solid phase devoid of galactose. The present invention now discloses that β-galactosidase is released from the sepharose solid phase during the incubation time required for activating Gc protein to MAF. Thus, while in the hitherto disclosed process the immobilized β-galactosidase is removed from the composition, the soluble enzyme or residues thereof can still contaminate the composition.

According to certain embodiments, the compositions of the present invention comprising GcMAF comprise less than 3% glycosidase enzymes out of the total protein content of said composition. According to other embodiments, the compositions comprise less than 2%, typically less than 1%, more typically less than 0.5% or 0.2% of the total protein content of the composition. Each possibility represents a separate embodiment of the present invention. As used herein, the term “percentage (%) of the total protein of the total protein content” refers to the weight/weight percentage of the glycosidase enzymes out of the total protein content.

According to certain embodiments, the immobilization solid phase of β-galactosidase which, according to the teaching of the present invention is devoid of the galactosidase substrate is composed of acrylic beads, capable of conjugating the enzyme. Several commercially available resins may be used for immobilization of the galactosidase enzymes, including, but not limited to Profinity epoxide (BioRad); Fractogel® Epoxy (Merck); and Eupergit® (BioChemika, Sigma).

In addition to β-galactosidase, at least one additional glycosidase enzyme may be used, depending on the Gc protein genotype. For activating Gc1f protein, β-galactosidase and sialidase are used to convert Gc1f to GcMAF. For activating Gc1s protein, β-galactosidase and mannosidase are used to convert Gc1s to GcMAF. As for β-galactosidase, each of the additional enzymes used may be immobilized on a solid phase. To avoid the presence of mannosidase and/or sialidase in the final composition according to the teachings of the present invention, each of the enzymes mannosidase and sialidase is immobilized on a substrate devoid of the enzyme substrate.

According to certain embodiments, the mannosidase or sialidase are immobilized on beads based on agarose.

Alternatively and additionally, the glycosidase enzymes, either soluble or immobilized, are removed from the GcMAF composition by several chromatography techniques and/or filtration and/or precipitation and/or centrifugation, separating the enzymes from the desired GcMAF protein.

Typical chromatography techniques include separation by size (size exclusion, also referred to as gel filtration); by charge (ion exchange chromatography), by hydrophobicity (hydrophobic interaction chromatography and reverse phase chromatography) and by bio-recognition (affinity chromatography).

According to certain embodiments, the process further comprises subjecting the GcMAF containing composition to ion exchange chromatography. According to typical embodiments, the chromatography is anion exchange chromatography. Various types of anion exchange resins can be used, including DEAE-Sephadex, QAE-Sephadex, DEAE-Sephacel, DEAE-cellulose, DEAE-Sepharose, Q-sepharose and the like.

The present invention now discloses that subjecting the GcMAF composition to anion exchange column further results in the separation of active GcMAF from non-active forms. Thus, the present invention provides means for achieving high yield isolation and purification of active GcMAF from various sources.

According to certain embodiments, the anion exchange resin is Q-Sepharose. Variety of conditions may be used in this particular step. According to certain embodiments, the anion exchange resin is first equilibrated with buffer solution having a pH between 4.5-9.5 and a conductivity of below 12.0 mS/cm. After the resin is equilibrated, the fraction containing GcMAF is adjusted to ion strength below 12 mS/cm by dilution and loaded on the anion exchange resin. These conditions of pH and conductivity allow the retention of GcMAF on the column, while the anion exchange medium is washed. The conductivity of the washing buffer (at a pH range of from 4.5 to 9.5) is increased during the washing. This increase provides suitable conditions such that no GcMAF is discarded in the flow through, to give maximal GcMAF yield.

The GcMAF is then eluted from the column. According to certain embodiments, elution is performed with a buffer solution having a pH between 4.5-9.5 and conductivity greater than 3 mS/cm.

According to other embodiments, the process further comprises subjecting the GcMAF composition to hydrophobic interaction chromatography, for example phenyl sepharose column.

According to certain embodiments, the hydrophobic interaction resin is phenyl-Sepharose. Variety of conditions may be used in this particular step. According to certain embodiments, the hydrophobic interaction resin is first equilibrated with buffer solution having a pH between 4.5-9.5 and a conductivity of above 15.0 mS/cm. After the resin is equilibrated, the fraction containing GcMAF is adjusted to ion strength above 15.0 mS/cm by dilution and loaded on the hydrophobic interaction resin. These conditions of pH and conductivity allow the retention of GcMAF on the column, while the hydrophobic interaction medium is washed. The conductivity of the washing buffer (at a pH of between 4.5-9.5) is decreased during the washing. This decrease provides suitable conditions such that no GcMAF is discarded in the flowthrough, to give maximal GcMAF yield.

The GcMAF is then eluted from the column. According to certain embodiments, elution is performed with a buffer solution having a pH between 4.5-9.5 and conductivity below 25 mS/cm.

The sterility of the compositions of the present invention is of major concern, as the product should be administered to humans for therapeutic purposes, in particular by intravenous administration. Although the serum source material is typically examined for the presence of contaminating viruses and a great effort is taken to exclude contaminated donor fractions, there is a need to further assure that the end product of the process would be virus-free.

Accordingly, the process of the present invention can further comprises viral removal and/or viral inactivation steps. Viral reduction can be accomplished by several processes, including nanofiltration; solvent/detergent treatment; iodine inactivation, e.g., treatment with an iodinated ion exchange matrix material such as iodinated SEPHADEX™ (as disclosed in PCT applications WO 97/48422 and WO 97/48482); treatment with Pathogen Inactivating Compounds; heat inactivation, gamma irradiation; or any other suitable virucidal process.

Lipid coated viruses are effectively inactivated by treatment with non-ionic biocompatible solvents and detergents. Methods for virus inactivation by solvent-detergent applications are described, for example, in EP 0131740. However, non-lipid coated viruses cannot be inactivated by solvent-detergent treatments, thus, other inactivation methodologies have to be used for their inactivation, including eliminating by physical means, e.g., the filtration of the composition through very small pore size filter so as to remove viruses by size exclusion (nanofiltration).

Following separation of the glycosidase enzymes from the active GcMAF and viral removal, the solution can be treated to reduce its water content and change the ionic composition by conventional means such as by diafiltration, ultrafiltration, lyophilization, etc., or combinations thereof.

According to certain embodiments, the composition comprising the purified GcMAF is dialyzed against PBS buffer by ultrafiltration with MWCO between 5-50 kDa. The GcMAF protein is then diluted with PBS to its final concentration and is filtered through 0.1 or 0.2 μm membrane to obtain sterile GcMAF composition.

According to additional aspect, the present invention provides a pharmaceutical composition comprising a therapeutically effective amount of the macrophage activating factor of the present invention, further comprising pharmaceutically acceptable diluent or carrier. According to preferred embodiments, the pharmaceutical composition is formulated for intravenous administration.

As used herein, the term “therapeutically effective amount” refers to an amount of a protein or protein formulation or composition which is effective to treat a condition in a living organism to whom it is administered over some period of time.

Pharmaceutical compositions of the present invention may be manufactured by processes well known in the art, e.g. by means of conventional mixing, dissolving, granulating, grinding, pulverizing, dragee-making, levigating, emulsifying, encapsulating, entrapping or lyophilizing processes. The purpose of a pharmaceutical composition is to facilitate administration of a compound to an organism.

Pharmaceutical compositions for use in accordance with the present invention thus may be formulated in conventional manner using one or more acceptable diluents or carriers comprising excipients and auxiliaries, which facilitate processing of the active compounds into compositions, which can be used pharmaceutically. Proper formulation is dependent on the route of administration chosen. According to typical embodiment the pharmaceutical compositions of the present invention are formulated for intravenous administration.

For intravenous injection, the compounds of the invention may be formulated in aqueous solutions, preferably in physiologically compatible buffers such as Hank's solution, Ringer's solution, or physiological saline buffer. Aqueous injection suspensions may contain substances, which increase the viscosity of the suspension, such as sodium carboxymethyl cellulose, sorbitol or dextran. Optionally, the suspension may also contain suitable stabilizers or agents to increase the stability and solubility of the compounds and to allow for compositions of highly concentrated solutions.

Alternatively, the active ingredient may be in powder form for reconstitution with a suitable vehicle, e.g., sterile, pyrogen-free water, before use.

Pharmaceutical compositions suitable for use in context of the present invention include compositions wherein the active ingredients are contained in an amount effective to achieve the intended purpose. More specifically, a therapeutically effective amount means an amount of a compound effective to prevent, alleviate or ameliorate symptoms of a disease of the subject being treated.

Determination of a therapeutically effective amount is well within the capability of those skilled in the art. According to certain embodiments, the GcMAF is administered at a dose of 100-500 ng/injection. A skilled artisan can determine the regime of administration according to parameters associated with the particular disease and stage to be treated as well as on characteristics of the treated individual (age, size, gender, etc.).

Toxicity and therapeutic efficacy of the compounds described herein can be determined by standard pharmaceutical procedures in cell cultures or experimental animals, e.g., by determining the IC50 (the concentration which provides 50% macrophage activation) and the LD50 (lethal dose causing death in 50% of the tested animals) for a GcMAF compound according to the present invention. The data obtained from these cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Depending on the severity and responsiveness of the condition to be treated, dosing can be determined. According to certain embodiments, the pharmaceutical composition comprising the GcMAF of the invention is administered via intravenous injection once a week for several weeks. The dosing may also be a single administration of a slow release composition, with course of treatment lasting from several days to several weeks or until cure is affected or diminution of the disease state is achieved.

The amount of a composition to be administered will, of course, be dependent on the subject being treated, the severity of the affliction, the manner of administration, the judgment of the prescribing physician, etc.

The following example is presented in order to more fully illustrate some embodiments of the invention. It should, in no way be construed, however, as limiting the broad scope of the invention. One skilled in the art can readily devise many variations and modifications of the principles disclosed herein without departing from the scope of the invention.

### EXAMPLES

### Example 1: GcMAF Production

Step 1: Enrichment—precipitation with Ammonium sulfate

Blood was obtained from healthy donors. Gc protein type of each blood sample was determined using specific primers in a PCR reaction and further by mass spectrometry. Typing was performed by BioGlobe GmbH Hamburg, Germany, based on the method described in Abbas et al. 2008 (Cancer Epidemiol Biomarkers Prey 17:1339-1343). Samples of donors homozygous to the Gc1f allele were taken for further processing. One out of 50 samples screened in 2009 and seven out of 105 samples screened in 2010 were identified as homozygous for Gc1f.

The serum fraction was isolated from the blood samples, and the Gc protein was precipitated from serum by 70% ammonium sulfate (AS). After centrifugation, the precipitated protein was dissolute in PBS and dialyzed against the same buffer having pH about 7.0-7.6.

Step 2: Capture—Vitamin D-sepharose affinity column

The protein was loaded on a 25-OH-vitamin D affinity column pre-equilibrated with TEST buffer (Tris, EDTA, Saline, Triton, pH 7.4). After washing with TEST buffer the protein was eluted by 6 M GuHCl. Fractions with peak absorbance at 280 nm were pooled and dialyzed against a phosphate buffer having pH 7.0.

Step 3: Purification—hydroxyapetite column

The protein was loaded on a hydroxyapatite column pre-equilibrated with phosphate buffer having pH 7.0. After washing with the same buffer the protein was eluted by a 10-200 mM linear gradient of phosphate. Fractions containing the protein were pooled.

Step 4: Gc activation (preparation of GcMAF)

The pooled protein fraction, containing Gc1f type only, was treated with β-galactosidase conjugated to acrylic beads (Profinity Epoxide, BioRad) for 1 hour in PBS pH 7.4 at 37° C. with gentle mixing. The enzyme conjugated to the acrylic beads was removed by centrifugation, and the protein fraction was collected. Next, the protein fraction was treated with Neuraminidase conjugated to agarose for 1 hour in 0.1 M NaAc 2 mM CaCl₂ pH 5.0 at 37° C. with gentle mixing and the conjugated enzyme was removed by centrifugation. The protein fraction, now containing GcMAF, was collected and filtrated through a 0.22 μm filter and stored at 4° C. The protein type was verified by ELISA and it quantity by Bradford assay.

Step 5: Final purification

GcMAF is processed on Ion Exchange (IEX) column (such as Q sepharose) and on Hydrophobic interaction chromatography (HIC) column (such as phenyl sepharose) to remove residual contaminating enzymes and non activated Gc protein. The composition is then examined to be essentially devoid of glycosidase enzymes by measuring glycosidase activity and/or presence of glycosidase using specific antibodies.

### Step 6: Formulation

The purified protein is dialyzed against PBS by ultrafiltration with MWCO between 10 kDa and 50 kDa. The protein is then diluted with PBS to its final concentration and filtered through 0.2 μm Millipore filter for sterilization.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. A composition comprising Gc protein-derived macrophage activating factor (GcMAF) wherein the composition is essentially devoid of glycosidase enzymes.

2. The composition of claim 1, wherein said composition comprises less than 3% glycosidase enzymes out of a total protein content of said composition.

3. The composition of claim 1, wherein the GcMAF comprises vitamin D-binding protein (Gc Protein) or a fragment thereof having an N-acetylgalactosamine group linked to an amino acid residue, and wherein said GcMAF comprises the amino acid sequence set forth in any one of SEQ ID NOs:1-3.

4. The composition of claim 3, wherein the N-acetylgalactosamine group is linked to the amino acid threonine at a position selected from the group consisting of position 418 and position 420.

5. The composition of claim 3, wherein the Gc protein fragment comprises the amino acid sequence corresponding to amino acids 400-435 of the Gc Protein.

6. The composition of claim 5, wherein the Gc fragment consists of the amino acid sequence set forth in SEQ ID NO:4 or SEQ ID NO:5.

7. The composition of claim 6, wherein the N-acetylgalactosamine group is linked to the amino acid threonine at a position selected from the group consisting of position 44 and position 46.

8. The composition of claim 1, wherein the Gc-protein or fragment thereof is obtained from blood serum or is produced from a cloned polynucleotide.

9. A process for producing a GcMAF composition essentially devoid of glycosidase enzymes, the process comprising the steps of:
(a) contacting Gc protein or an active fragment thereof in vitro with the glycosidase enzyme β-galactosidase or with β-galactosidase in combination with at least one additional glycosidase enzyme, wherein each of the glycosidase enzymes is immobilized on a solid phase devoid of said enzyme substrate, to obtain Gc-macrophage activating factor (GcMAF); and
(b) removing the immobilized enzyme from the GcMAF, thereby obtaining a GcMAF composition essentially devoid of glycosidase enzymes.

10. The process of claim 9, wherein said additional glycosidase enzyme is mannosidase or sialidase.

11. The process of claim 9, wherein the GcMAF comprises Gc Protein or a fragment thereof having an N-acetylgalactosamine group linked to an amino acid residue, and wherein said Gc protein comprises the amino acid sequence set forth in any one of SEQ ID NOs:1-3.

12. The process of claim 11, wherein the Gc protein fragment consists of the amino acids sequence set forth in SEQ ID NO:4 or SEQ ID NO:5.

13. The process of claim 12, wherein the N-acetylgalactosamine group is linked to the amino acid threonine at a position selected from the group consisting of position 44 and position 46.

14. The process of claim 9 wherein the Gc-protein or fragment thereof is obtained from blood serum or is produced from a cloned polynucleotide.

15. The process of claim 9, further comprising at least one step selected from the group consisting of subjecting the GcMAF containing composition to ion exchange chromatography, subjecting the GcMAF containing composition to hydrophobic interaction chromatography and a combination thereof.

16. The process of claim 15, wherein the ion exchange chromatography is an anion exchange chromatography.

17. The method of claim 9, wherein the β-galactosidase is immobilized on a solid phase comprising acrylic beads.

18. A composition comprising GcMAF produced by the method of claim 9, wherein the composition comprises less than 3% glycosidase enzymes out of a total protein content of said composition.

19. A pharmaceutical composition comprising a therapeutically effective amount of a composition comprising the macrophage activating factor of claim 1 and a therapeutically acceptable diluent or carrier.

20. A pharmaceutical composition comprising a therapeutically effective amount of a composition comprising the macrophage activating factor of claim 18 and a therapeutically acceptable diluent or carrier.

21. The pharmaceutical composition of claim 19, wherein the pharmaceutical composition is formulated for intravenous administration.

22. A method for inducing macrophage activation in an individual in need thereof comprising administering to the individual the pharmaceutical composition of claim 21.

23. A method for treating cancer, comprising administrating to a subject in need thereof a therapeutically effective amount of the pharmaceutical composition of claim 21.

24. A method for treating an HIV-infected patient, comprising administrating a therapeutically effective amount of the pharmaceutical composition of claim 21 to the HIV-infected patient in need thereof.

25. The method of claim 23, wherein the cancer is associated with elevated levels of alpha-N-acetylgalactosaminidase (Nagalase).

26. The method of claim 23, wherein the cancer is selected from the group consisting of breast cancer, prostate cancer, colorectal cancer, liver cancer, lung cancer, head/neck cancer, brain cancer, kidney cancer, bladder cancer, stomach cancer, uterus cancer, ovarian cancer, skin cancer, fibrosarcoma, mesothelioma, leukemia and melanoma.

27. The pharmaceutical composition of claim 20, wherein the pharmaceutical composition is formulated for intravenous administration.

28. The method of claim 24, wherein the HIV is associated with elevated levels of alpha-N-acetylgalactosaminidase (Nagalase).
